(19) United States
(12) Patent Application Publication
(10) Pub. No.: US 2024/0390679 A1
(43) Pub. Date: Nov. 28, 2024

(54) INTRAVASCULAR IMPLANT FOR STIMULATING OF BLOOD PRESSURE

(71) Applicant: BIOTRONIK SE & Co. KG, Berlin (DE)

(72) Inventors: Christian MOSS, Falkensee (DE); Franziska WEGERICH, Berlin (DE)

(73) Assignee: BIOTRONIK SE & Co. KG, Berlin (DE)

(21) Appl. No.: 18/695,996

(22) PCT Filed: Oct. 14, 2022
(86) PCT No.: PCT/EP2022/078633

§ 371 Date: Mar. 27, 2024

(30) Foreign Application Priority Data
Oct. 22, 2021 (EP) 21204129.7

(51) Int. Cl. A61N 1/36; A61N 1/05; A61N 1/372; A61N 1/378

(52) U.S. Cl. CPC A61N 1/36139; A61N 1/0551; A61N 1/36114; A61N 1/36125; A61N 1/36153; A61N 1/36157; A61N 1/37211; A61N 1/3787

(57) ABSTRACT

An implantable sensor device comprising means for determining a blood pressure and means for stimulating at least one nerve cell, based at least in part on the determined blood pressure. Further aspects relate to a method carried out by such a device and a computer program.

### CROSS-REFERENCE TO RELATED APPLICATIONS

This application is the United States National Phase under 35 U.S.C. § 371 of PCT International Patent Application No. PCT/EP2022/078633, filed on Oct. 14, 2022, which claims the benefit of European Patent Application No. 21204129.7, filed on Oct. 22, 2021, the disclosures of which are hereby incorporated by reference herein in their entireties.

### TECHNICAL FIELD

The present invention generally relates to a sensor device and a method and a computer program for operating such a device, particularly for stimulating a nerve cell based on a determined blood pressure.

### BACKGROUND

Many types of treatments are currently known for patients who may suffer from various types of syncopes. A syncope is a loss of consciousness which may be associated with a decrease of blood flow to the brain resulting from low blood pressure. The blood pressure drop may have various medical reasons and root causes. Types of syncopes may be an orthostatic reflex-syncope, a vasovagal reflex-syncope, or a situational syncope, etc. For example, reflex-syncopes may be the most common types of syncopes (e.g., 50% of all syncopes) and may affect at least 1 in 1000 people per year. Even though syncopes themselves may not be considered life threating conditions, a person experiencing a syncope may be significantly harmed or may even die from the effects resulting from the sudden loss of consciousness (e.g., when driving in a car, etc.). For example, the loss of consciousness may cause the person to fall uncontrollably to the ground potentially causing significant physical injury. In addition, the unconscious state may increase the risk of hypoxia due to the uncontrolled state of the person's respiratory system (e.g., a tongue position not favorable to the breathing process, pulmonary aspiration of vomit etc.).

Currently, syncope prevention may comprise treating the patient with medication which may improve the regulation of the patient's blood pressure. This approach may enable managing some types of syncopes in certain patient groups.

However, this type of treatment has various limitations. The medication may only be effective for a specific type of syncope (e.g., only an orthostatic syncope) therefore limiting the range of treatment. This therapy approach may further suffer from the common drawbacks associated with medication treatment of patients. For example, some patients may experience side effects or intolerances to the drug treatment. In some patients, the medication may not be effective to prevent syncopes which causes them to continuingly experience syncopes.

Therefore, there is a need to improve the prevention of syncopes.

The aspects described herein address the above need at least in part.

The present disclosure is directed toward overcoming one or more of the above-mentioned problems, though not necessarily limited to embodiments that do.

### SUMMARY

A first aspect relates to an implantable sensor device which may comprise means for determining a blood pressure. The implantable sensor device may further comprise means for stimulating at least one nerve cell, based at least in part on the determined blood pressure.

The underlying idea is based on the medical mechanism that a stimulation of a nerve cell may influence the blood pressure. The inventive device may enable a fully intrinsic (i.e., in situ) approach to purposefully stimulate the at least one nerve cell depending on the blood pressure wherein the implantable sensor device may fully reside in a patient. This may benefit a variety of patients who are at risk of having a syncope which may be associated with a deviation in blood pressure, and by stimulation based on the determined blood pressure, a sudden drop of blood pressure may be prevented or at least counteracted such that syncope may effectively be prevented.

In one regard, the present invention may resolve the drawbacks of medication treatment of patients in the syncope risk group. The use of the sensor device may overcome the necessity to administer a medication (e.g., a drug) to the patient for treating/preventing a syncope. Hence, the sensor device may allow a syncope therapy for patients whose bodies may not effectively react to the medication for treating/preventing syncopes. The present invention may enable these patients to actually undergo a syncope therapy which may significantly reduce the occurrences of syncopes for these patients and may thus significantly increase their perceived living quality and overall health. Furthermore, since the syncope therapy achievable by the device may not require any drug intake, side effects and/or intolerances arising from medication treatment may be avoided in general by the inventive device.

In addition, the device may overcome extrinsic solutions for determining the blood pressure (e.g., by a sphygmomanometer) and for stimulating the at least one nerve cell (e.g., via extrinsic electrode needles attached thereto by a percutaneous procedure), as it relates to an implantable device.

The sensor device is configured for implanting into a blood vessel. In addition, the sensor device is configured for implantation via a catheter.

Hence, the sensor device may be operably configured as a (single) intravascular implant. For example, the sensor device may be configured for implanting into a renal artery, aorta and/or an area in the crossing between the renal artery and aorta. The position in the area of the renal artery and/or aorta may be a beneficial implant location for the sensor device due to the comparatively wide blood vessel dimensions which may enable a mechanically stable connection. This may ensure a reliable fixation of the sensor device which may lead to a reliable readout of the blood pressure, a reduction of design complexity and/or a stable stimulation position. Notably, the sensor device may be configured for implanting into any artery, elastic artery, distributing artery, vein, arterioles, capillaries, venules, sinusoids of a patient.

In some examples the means for stimulating may be configured for fixing the sensor device to the blood vessel. This may be highly beneficial since two functions (i.e., mechanical fixation to the blood vessel and stimulating the at least one nerve cell) needed for a proper operation of the sensor device may be implemented by the means for stimulating. Hence, extra hardware may not be required which may significantly reduce design complexity and/or enable an easier implant procedure for medical personnel. The means for stimulating may be configured such that a mechanically stable fixation to the blood vessel may correlate with an optimal position for stimulating the at least one nerve cell (and/or vice versa).

In an example, the means for determining may be a system for sensing and/or determining the blood pressure. For example, the system may comprise a sensor unit, a sensor and/or sensing element for sensing the blood pressure signal. The blood pressure may be determined by the system from the sensed blood pressure signal by applying signal processing to the sensed signal. The signal processing may be carried out by hardware (e.g., by a signal processing unit, a microcontroller, a microprocessor, an ASIC, an embedded system, etc.) and/or software comprised in the system and/or the sensor device.

In an example, the means for stimulating of the inventive sensor device may comprise one or more elements which may excite the at least one nerve cell. The excitation of the at least one nerve cell may be based at least in part on releasing electromagnetic energy/power, e.g., electric energy/power, etc., by the means for stimulating (e.g., by its one or more elements) which may be coupled to the at least one nerve cell. The sensor device may thus be fully equipped with neurostimulation capabilities. For example, the means for stimulating may comprise a stimulator (e.g., with one or more electrodes serving as the interface to the environment), a control unit (e.g., a microcontroller, a microprocessor, an ASIC, etc.), a power supply and/or a stimulation unit (a power-electronic-circuitry) to deliver a defined amount of electrical energy/power (e.g., over the electrode) to the at least one nerve cell. The sensor device may be configured such that the means for stimulating are in the vicinity of (or in direct contact with) the at least one nerve cell for an improved coupling of the excitation output of the means for stimulating to the at least one nerve cell. The at least one nerve cell may comprise any cell of a nervous system, a neuron, a nerve fiber, a dendrite and/or an axon etc.

In an example, the means for determining and means for stimulating of the implantable sensor device may be not share elements with each other. For example, the means for determining may not share hardware with the means for stimulating. The means for determining and the means for stimulating may be separate units which may only be communicatively coupled to each other (e.g., over a wired connection, wirelessly, etc.). The means for determining and means for stimulating may thus be arranged in different areas of the body of the patient and/or may be separately configurable. To illustrate an example, the means for determining may be a blood pressure sensor unit (e.g., with its own power supply, signal processing unit, microcontroller etc.) configured for implanting into area A of a patient. The means for stimulating may be a separate neurostimulation unit (e.g., with its separate stimulator, power-electronic-circuitry and/or power supply, etc.) which may be configured for implanting into area B of the patient for stimulating the at least one nerve cell. In this example, the blood pressure sensor unit may communicatively transmit the determined blood pressure in area A, wherein the means for stimulating may stimulate the at least one nerve cell depending on the determined blood pressure.

In an example, the means for determining and the means for stimulating may share one or more elements with each other. For example, the means for determining may share hardware (e.g., a power supply, a microcontroller, etc.) and/or software with the means for stimulating. In another example, one main unit (i.e., one local entity) may comprise some elements or all elements of the means for determining and/or the means for stimulating (e.g., inside one casing of the main unit). To illustrate an example, some elements of the means for stimulating (e.g., a stimulator with an electrode) may be positioned outside of the main unit with a wired connection to other elements of the means for stimulating which may reside inside the main unit (e.g., a microcontroller). Some elements of the means for determining (e.g., a sensor unit) may be positioned outside of the main unit with a wired connection to other elements of the means for determining which may reside inside the main unit (e.g., the microcontroller, wherein the microcontroller is shared with the means for stimulating). The implantable sensor device may be configured such that the means for determining and the means for stimulating may communicate with each other (e.g., over a wired connection and/or a wireless connection).

In another example the sensor device may comprise separate means for fixing the sensor device to the blood vessel. This may be beneficial for enabling various types of mechanical fixation mechanisms which may be required to ensure a stable fixation to a broad range of blood vessel types. Further, the means for stimulating may thus not need to provide a mechanical fixation function which may enhance the stimulation capabilities of the sensor device. For example, a wider degree of freedom of the design of the means for stimulating may be enabled which may be beneficial to the stimulation range and/or stimulation interface (e.g., stimulating a nerve cell in a further distance to the sensor device, optimized stimulation design not restrained by the mechanical fixation function, etc.).

In another example the means for stimulating may be configured for being electrically coupled to the blood vessel. Hence, when the device is implanted into a blood vessel, it may couple electrical excitations (e.g., electrical energy/power) to the blood vessel. For example, the means for stimulating may be configured for being electrically coupled to a wall, a layer of a wall and/or a tissue of the blood vessel. For example, the means for stimulating may be configured for being electrically coupled to a basement membrane, a tunica interna, a tunica intima, a tunica media, a tunica externa, a tunica adventitia and/or a connective tissue of a blood vessel or any combination thereof. It may also be configured for being electrically coupled to the fluid of the blood vessel and/or cells/plasma transported in the blood vessel. In an example, the means for stimulating comprise an electrode with an electrically conductive surface as an interface for the electrical coupling to the blood vessel (e.g., to the wall, tissue and/or fluid thereof, etc.). For example, the electrode may be in a direct contact with the inner wall of the blood vessel (e.g., directly pressed against it). The electrode may be also be configured to be in direct contact with a basement membrane, a tunica intima, a tunica media, a tunica externa, a tunica adventitia and/or a connective tissue of a blood vessel or any combination thereof. In another example, it may be in an indirect contact with the blood vessel, namely freely residing in the blood vessel (e.g., surrounded by the fluid of the blood vessel). It may also be conceivable that some parts of the electrode may be in a direct contact with one or more layers of the blood vessel wall, while other parts of the electrode may be in an indirect contact with the blood vessel (e.g., freely residing therein). The means for stimulating (e.g., its electrode) may be configured such that its electrical coupling to the blood vessel facilitates an enhanced electrical coupling (e.g., such that a high efficiency factor is reached of the coupled electrical energy). For example, the means for stimulating may be configured to reduce (unnecessary) energy loss during the coupling to the blood vessel (e.g., loss by heat absorption) and/or reduce parasitic electrical effects (e.g., parasitic capacities, parasitic inductances which may impact static, pulsed and/or alternating current based electrical couplings). For example, the means for stimulating (e.g., its electrode) may have an area dimension, a shape, a surface property and/or may comprise a certain material which may facilitate an optimized electrical coupling. For example, the electrode may be a conductive (e.g., metallic) wire with a specific segment of the wire bent into close (or direct) contact to the wall of the blood vessel. Other examples may be a circular shaped electrode, a rectangular shaped electrode, a needle shape electrode, a porous electrode surface and/or an electrode with a fractal coating. The fractal coating may be a conductive material in an irregular manner deposited onto the sensor surface. This may increase the electrochemically active surface area which may improve the electrical coupling to the blood vessel.

In another example, the means for stimulating may be configured to stimulate the at least one nerve cell by applying an electrical stimulus to the blood vessel. The means for stimulating may thus be configured for transvascular stimulation of the at least one nerve cell wherein applying an electrical stimulus to the blood vessel may be routed to the at least one nerve cell. For example, the sensor device may be configured such that the electrical stimulus is conducted from the blood vessel over the body's tissue (e.g., over the blood vessel layers, muscle tissue, nerve cells, etc.) to the at least one nerve cell. In an example, the sensor device may be configured such that the means for stimulating may stimulate at least one nerve cell which is in the vicinity of the location of the electrical stimulus applied to the blood vessel by the means for stimulating. An electrical stimulus may be any form of electrical excitation to the blood vessel with an electrical energy, electrical power, electrical current and/or electrical voltage. For example, the electrical stimulus may be a pulsed electrical (current and/or voltage) stimulation (e.g., with a certain pulse duration, duty cycle, pulse amplitude, pulse frequency, a FWHM characteristic, etc.), a constant current, a constant voltage, an alternating current (e.g., AC), and/or an alternating voltage. This approach may be beneficial since it overcomes the necessity to directly contact the at least one nerve cell for stimulating it. The inventive concept may thus enable that the implantable sensor device may be situated in a blood vessel with full neurostimulation capabilities without requiring external leads directly contacting the at least one nerve cell. This may significantly simplify implant procedures for medical personnel, reduce system complexity and/or reduce potential medical complications arising from directly contacting the at least one nerve cell (e.g., nerve damage, infections, etc.).

In another example the means for stimulating may comprise a first portion configured for being electrically coupled to a first vessel part of the blood vessel. It may further comprise a second portion configured for being electrically coupled to a second vessel part of the blood vessel. Further, the means for stimulating may be configured to stimulate the at least one nerve cell by applying an electrical current and/or a voltage between the first portion and the second portion. The first portion and the second portion may thus span a vessel segment through the blood vessel locally defined by the first vessel part and the second vessel part. This achieves that the electrical coupling may be narrowed to the vessel segment and/or vessel segment area. For example, the means for stimulating may be configured such that the first and second portion may be controlled as separate terminals of an electrical stimulus. The means for stimulating may form a closed loop (i.e., an electrical circuit) through the body of a patient extending from the first portion over the vessel segment to the second portion. The electrical output of the first and second portion may be separately controlled by the sensor device (e.g., the first portion may be ground, the second portion may apply a voltage/current). As an example, this enables that a defined electrical current may flow between the first portion and second portion through the vessel segment. The first portion and the second portion may be mechanically fixed to the blood vessel to define a constant vessel segment over a prolonged period of time (e.g., the means for stimulating may be configured such that the first and second portion may be directly pressed against the inner wall of the blood vessel).

In another example, the first and second portion may be configured to function as two separate stimulation points. For example, the first and second portion may be separate electrodes which are electrically decoupled for applying separate electrical stimuli to the blood vessel.

In an example, the at least one nerve cell may be a cell of an aorticorenal ganglion. For example, the sensor device may be configured for implanting into a blood vessel in the vicinity of the aorticorenal ganglion (e.g., a renal artery, aorta) wherein an electrical stimulus to the blood vessel may stimulate at least one cell of the aorticorenal ganglion. The means for stimulating may also be configured for stimulating a plurality of cells of an aorticorenal ganglion, every cell of an aorticorenal ganglion, a plurality of cells of aorticorenal ganglions and/or a plurality of aorticorenal ganglions.

In another example, the means for determining may comprise a membrane. For example, the means for determining may comprise a blood pressure sensor with a flexible membrane which may be configured such that it is exposed to the blood pressure. For example, the means for determining may be configured such that the membrane may be coupled to the encasing of a main unit (e.g., which may encompass most of the sensor device's hardware (e.g., power supply, microcontroller, etc.)). A change in blood pressure may lead to a corresponding change of the bending characteristics of the membrane. The membrane may, for example, be coupled to an oil-based reservoir comprised in the sensor device wherein the bending of the membrane may be coupled to the oil-based reservoir to effectuate a change in the pressure of the oil-based reservoir. The means for determining may be configured to determine the blood pressure based at least in part on the pressure in the oil-based reservoir. The membrane may be of a material inert to the biological environment (e.g., a titanium-based membrane).

In another example, the sensor device may be configured such that stimulating the at least one nerve cell effectuates a change in the blood pressure. For example, the stimulation characteristics may be configured such that by stimulating the at least one nerve cell with a specific stimulus (defined by certain stimulation characteristics) the blood pressure of the patient may increase (or decrease). The sensor device may thus be configured to cause a medically relevant change in blood pressure for the patient. For example, the means for stimulating may be configured such that the specific stimulus comprises a specific electrical excitation (e.g., with a specific electrical energy, specific electrical power, specific electrical current and/or specific electrical voltage) which may effectuate an increase in the blood pressure. To illustrate an example, the electrical excitation may comprise one or more specific current pulses with a frequency in a range spanned between values of 5 Hz, 10 Hz, 15 Hz and/or 20 Hz with a current amplitude in a range spanned between values of 5 mA, mA, 15 mA, 20 mA, 25 mA, 30 mA and/or 50 mA or any combination thereof. However, in other examples, other frequencies may be used, and the electrical stimulus may comprise one or more electrical current and/or voltage pulses with a frequency below 1 Hz, 50 Hz, 100 Hz, 500 Hz, 1 MHz, 100 MHz and/or 1 GHz. Further, the one or more current pulses may also comprise different amplitudes, for example, current pulses with a current amplitude below 100 mA, 150 mA, 200 mA, 300 mA, 400 mA, 500 mA, 800 mA may be provided. In case of voltage pulses, one or more voltage pulses may comprise a voltage pulse with a voltage amplitude below 1 pV, 1 mV, 10 mV, 100 mV, 1 V, 12 V, 24 V, 100 V, 650V, 800V and/or 1 kV.

In another example the sensor device may be configured to apply one or more stimulations to the at least one nerve cell if the determined blood pressure is below (or above) a predetermined threshold. Hence, the sensor device may enable a physical increase (or decrease) in blood pressure when the blood pressure of the patient is below (or above) the predetermined threshold. This approach may enable an efficient system mechanism to regulate a patient's blood pressure since the sensor device only needs to compare the determined blood pressure with the predetermined threshold which may require a minimal calculation effort. Hence, the system complexity and/or energy consumption of the sensor device may be significantly reduced (e.g., by reduced hardware/software requirements). For example, the reduced energy consumption (e.g., due to reduced computational needs) may enable a smaller built of a power supply of the sensor device which may enable a smaller built of the sensor device itself.

The predetermined threshold may be configured such that the one or more stimulations may effectuate an increase in blood pressure before a syncope (i.e., a loss of consciousness) is reached due to the blood pressure drop. The predetermined threshold may be based on a medically relevant safety margin which may be associated with various factors. For example, the predetermined threshold (e.g., a variable p_{TH}) may be configured to take into account a predetermined time delay (e.g., a time t_{DEL}) which may be the time it takes to effectuate a blood pressure increase after a stimulation of the at least one nerve cell. Further, the predetermined threshold may be based on a critical blood pressure associated with a syncope (e.g., a blood pressure p_{CRIT}) and/or various other characteristics (e.g., stimulation characteristics, implant location, patient parameters, etc.). Notably, the predetermined threshold may be a function of the time delay, the critical blood pressure and/or the various other characteristics (e.g., p_{TH}=f(t_{DEL}, p_{CRIT}, . . . )).

In another example, the sensor device may be further configured to apply one or more stimulations to the at least one nerve cell such that the blood pressure is controlled to be between a predetermined first blood pressure and a predetermined second blood pressure. For example, the sensor device may be configured to implement a control system to regulate the blood pressure such that it remains in a certain range (e.g., a range safe for the patient's wellbeing). For example, this may ensure that the blood pressure does not significantly increase to a medically critical level (e.g., above the predetermined second blood pressure) while ensuring the blood pressure does not drop significantly below a medically critical level (e.g., below the predetermined first blood pressure). The sensor device may be configured such that the blood pressure is controlled in an autarkic (e.g., automatic) manner so that the blood pressure may be maintained in the range of the second blood pressure and first blood pressure over a prolonged period of time without manual intervention (e.g., over a period of 1 month, 1 year, 10 years, etc.).

To illustrate an example, the sensor device may be configured to implement a control system for controlling the blood pressure. The control system may comprise the blood pressure as the process variable, a predetermined blood pressure value as the set point, wherein the system input to the control system may be provided by the means for stimulating (e.g., the system input may be an electrical stimulus to the blood vessel). The system input may be based at least in part on comparing the blood pressure with the predetermined blood pressure value.

In another example the sensor device may be further configured to stimulate the at least one nerve cell such that a syncope is prevented and/or treated. For example, the sensor device may be configured to determine an emerging syncope and/or determine if a syncope is present (i.e., patient is unconscious due to a blood pressure change), and subsequently apply a corresponding stimulation for effectuating a blood pressure change. The sensor device may detect, if certain characteristics of the determined blood pressure correlate with a likelihood of an emerging and/or present syncope. This may be based at least in part on the rate of a blood pressure change (e.g., a derivative thereof), a determined baseline of the blood pressure, and/or a medically low blood pressure over a certain time period. The stimulation to the at least one nerve cell may be specific depending on whether an emerging syncope or a present syncope was determined by the sensor device. For example, the sensor device may be configured such that if an already unconscious patient was determined, the corresponding stimulation by the sensor device may increase the blood pressure at a first rate over a first time. If an emerging syncope was determined by the sensor device, it may be configured such that the corresponding stimulation of the sensor device may increase the blood pressure at a second rate over a second time. For example, it may be medically necessary that an already unconscious patient requires a slower increase of blood pressure (and/or different stimulus to the at least one nerve cell), than a patient experiencing an emerging syncope (e.g., which may require a short but strong increase in blood pressure to prevent it).

In another example the sensor device may be configured for wireless charging. For example, the sensor device may comprise a charging unit which may comprise a coil structure, as well as a rechargeable battery. The sensor device may be charged over a (electro)magnetic field coupled to the coil structure. Hence, the sensor device may be configured for inductive charging. The (electro)magnetic field for charging may be an alternating (electro)magnetic field and/or may originate from an external device (e.g., a manual device operated by the patient). The device longevity of the sensor device may be significantly increased with the wireless charging functionality. For example, this may enable that the sensor device can be charged without significant medical impact on the patient. It may thus be avoided or significantly delayed, that a battery change via surgery (e.g., explanting/implanting procedures by medical personnel) is necessary which may significantly reduce the medical stress and/or medical complications on the patient (e.g., infections, scar tissue, etc.) arising from surgery.

In another example the sensor device may be configured for communication with an external device. For example, the sensor device may comprise a communication unit which may comprise a sending, receiving and/or transceiving unit for communication with the external device. The external device may be a manual device operated by the patient, medical personnel and/or technical personnel, etc. for reading out data of the sensor device (e.g., blood pressure values, etc.) and/or sending data to the sensor device (e.g., calibration, programming instructions, control data, etc.). For example, the sensor device may be configured to automatically send a notification and/or alert if a critical power supply status has been reached (e.g., the battery of the sensor device needs to be charged).

A second aspect relates to a method carried out by an implantable sensor device which may comprise the following steps: determining a blood pressure and/or stimulating at least one nerve cell, based at least in part on the determined blood pressure.

In another example the method may be carried out by the implantable sensor device which may be configured for implanting into a blood vessel.

A third aspect relates to a computer program which may comprise instructions to perform the method as outlined herein, when the instructions are executed by the implantable sensor device. In an example the computer program instructions may be stored on a non-volatile memory. For example, the computer program may be stored on a sensor device as described herein, which may comprise means to execute the computer program instructions. The computer program may allow an autarkic, automated implementation of the aspects described herein. Consequently, technical intervention from medical staff and the patient may be minimized.

In the following, further method examples are described.

A further example relates to a treatment method, e.g., for treating patients with increased risk for a syncope, which may comprise determining a blood pressure of the patient and/or stimulating at least one nerve cell of the patient, based at least in part on the determined blood pressure. The method may be applied to prevent and/or treat a syncope of a patient, for example, and/or otherwise to prevent undesirable blood pressure values. The blood pressure may be determined internally (e.g., from within the patient) and/or externally (e.g., over the external circumference of the patient's arm). The method may further comprise diagnosing a syncope and/or an emerging syncope based at least in part on the determined blood pressure of the patient. For example, if a risk of an emerging syncope may be present (e.g., due to a specific blood pressure characteristic) the at least one nerve cell may be stimulated such that the syncope is prevented.

In another example the method for treating patients may additionally or alternatively comprise determining a syncope status of the patient and/or stimulating the at least one nerve cell of the patient, based at least in part on the determined syncope status of the patient (which may not necessarily comprise determining a blood pressure). The syncope status of the patient may comprise a risk group of the patient, a body position of the patient, a patient input (e.g., patient communicates that he/she is about to change a body position), a conscious state of the patient, an unconscious state of the patient. The status of the patient may be determined manually by the patient and/or another person (e.g., medical personnel, etc.). The stimulating of the at least one nerve cell may be triggered automatically or manually by the patient and/or another person (e.g., medical personnel, etc.). For example, if a syncope status of a patient comprises that the patient is in a specific syncope risk group and the patient knows he/she is about to stand up, the patient may manually trigger stimulating the at least one nerve cell such that a (possible) syncope may be prevented when he/she stands up. In another example, the medical personnel may determine an unconscious state of the patient (e.g., by basic observation, questions, etc.) and subsequently manually trigger stimulating the at least one nerve cell of the patient to treat the syncope.

In another example, the method for treating patients may further comprise implanting a sensor device for determining the blood pressure and/or stimulating the at least one nerve cell. The sensor device may thus be configured to implement the method internally without requiring external feedback/features outside of the patient's body for determining and/or stimulating. The sensor device may be configured as outlined herein.

In another example, the sensor device may be configured for implanting into a blood vessel.

In another example, the stimulating may comprise stimulating by means for fixing the sensor device to the blood vessel.

In another example, the stimulating may comprise applying an electrical stimulus to the blood vessel.

In another example, applying the electrical stimulus to the blood vessel may comprise applying the electrical stimulus in a vicinity of an aorticorenal ganglion.

In another example, the electrical stimulus may be adapted to effectuate a change in the blood pressure.

In another example of the treatment method, it may further comprise applying one or more stimulations to the at least one nerve cell if the determined blood pressure is below a predetermined threshold and/or applying one or more stimulations to the at least one nerve cell such that the blood pressure is controlled to be between a predetermined first blood pressure and a predetermined second blood pressure and/or stimulating the at least one nerve cell such that a syncope is prevented.

It is noted that the method steps as described herein may include all aspects described herein, even if not expressly described as method steps but rather with reference to an apparatus (or device). Moreover, the devices as outlined herein may include means for implementing all aspects as outlined herein, even if these may rather be described in the context of method steps.

Whether described as method steps, computer program and/or means, the functions described herein may be implemented in hardware, software, firmware, and/or combinations thereof. If implemented in software/firmware, the functions may be stored on or transmitted as one or more instructions or code on a computer-readable medium. Computer-readable media include both computer storage media and communication media including any medium that facilitates transfer of a computer program from one place to another. A storage medium may be any available media that can be accessed by a general purpose or special purpose computer. By way of example, and not limitation, such computer-readable storage media can comprise RAM, ROM, EEPROM, FPGA, CD/DVD or other optical disk storage, magnetic disk storage or other magnetic storage devices, or any other medium that can be used to carry or store desired program code means in the form of instructions or data structures and that can be accessed by a general-purpose or special-purpose computer, or a general-purpose or special-purpose processor. The control unit as described herein may also be implemented in hardware, software, firmware, and/or combinations thereof, for example, by means of one or more general-purpose or special-purpose computers, and/or a general-purpose or special-purpose processors.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

In the following, the Figures of the present disclosure are listed:

FIG. 1: Schematic representation of an exemplary embodiment of a sensor device according to the present invention.

FIG. 2: Schematic representation of the anatomy in the kidney area of a human.

FIG. 3: Schematic representation of an exemplary embodiment of a sensor device according to the present invention when implanted into a blood vessel in the kidney area of a human.

FIG. 4: Block diagram representing the functional relations of parts of an exemplary embodiment of a sensor device according to the present invention.

### DETAILED DESCRIPTION

Subsequently, presently preferred embodiments will be outlined, primarily with reference to the above Figures. It is noted that further embodiments are certainly possible, and the below explanations are provided by way of example only, without limitation.

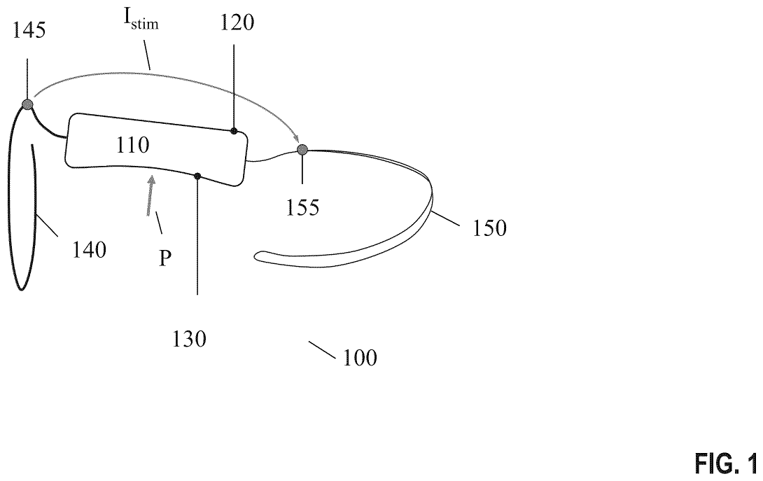

FIG. 1 shows a schematic representation of an exemplary embodiment of a sensor device 100 according to the present invention.

The sensor device 100 may comprise a main unit 110. The main unit 110 may encompass various components inside a casing 120, wherein the casing 120 may be of a material chemically durable with respect to an organic and/or aggressive environment. For example, the solid casing 120 may be made of titanium and/or a titanium compound and/or comprise a titanium-based coating to be inert when exposed to an organic/aggressive environment (e.g., blood and/or other organic fluids/tissue, e.g., nerve cells, acid, etc.). The casing of the main unit 110 may thus not significantly chemically react with an organic/aggressive environment which may minimize the influence of the casing onto the organic/aggressive environment and/or may minimize the deteriorating effects of the organic/aggressive environment onto the casing 120 and the components therein.

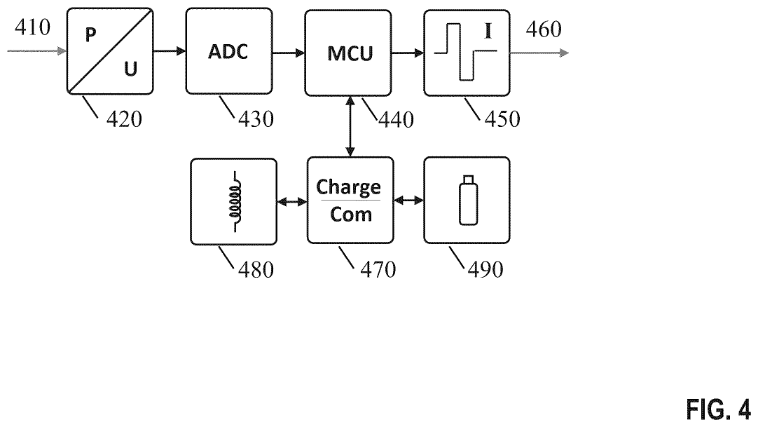

In an example, as seen in FIG. 1, the main unit 110 may comprise a membrane 130 which may bend under the influence of a pressure P of the surrounding environment. The membrane 130 may be of a material which is chemically durable regarding an organic and/or aggressive environment (e.g., titanium and/or a titanium compound and/or comprise a titanium-based coating), as outlined for the casing 120. The casing 120 may comprise an opening, wherein the membrane 130 may be fixedly mounted at its outer edge to the edges of the opening of the casing 120 such that the main unit 110 and its inner components are hermetically sealed from the environment. The membrane 120 may thus freely bend in the area defined by the opening in the casing 120 under the pressure P of the environment. This may enable an elastic coupling of the pressure P onto the inner parts of the main unit 110 without chemically exposing the inner components of the main unit 110 to the outer environment (e.g., to blood). The membrane 120 may be part of a blood pressure sensor 420 (functionally outlined in FIG. 4), wherein a separate sensor portion may be formed inside the main unit 110 in the area of the membrane 120. One or more parts of the sensor portion may be filled with an oil to couple the pressure P to a sensory chip which may sense and/or determine an absolute pressure and/or a relative pressure associated with the pressure P. Many types of sensory chips for sensing/determining the absolute and/or relative repressure may be conceivable (e.g., a piezoresistive pressure sensor, a piezoelectric pressure sensor, a capacitive pressure sensor, an electromagnetic pressure sensor, a strain-gauge, an optical pressure sensor, a resonant frequency pressure sensor, a thermal pressure sensor, etc.).

In another example, the main unit 110 may not comprise a membrane 130. It may be conceivable, that the pressure P may be sensed over a pressure sensor 420 which may not require a membrane (e.g., via an optical based sensor) wherein the pressure sensor 420 may be comprised by the main unit 110 (e.g., fixedly mounted to the outside of the casing 120). In another example, the pressure sensor 420 may be a separate external sensor unit which is may be communicatively coupled to the main unit 110 (e.g., over a wire and/or wirelessly). The separate external sensor unit may be any type of pressure sensor 420 for sensing/determining the absolute and/or relative pressure configured for an organic/aggressive environment (e.g., a piezoresistive pressure sensor, a piezoelectric pressure sensor, a capacitive pressure sensor, an electromagnetic pressure sensor, a strain-gauge, an optical pressure sensor, a resonant frequency pressure sensor, a thermal pressure sensor, etc.).

The main unit 110 may comprise further components which are functionally outlined in detail in FIG. 4. For example, the main unit 110 may comprise an analog-to-digital converter 430, a microcontroller 440, a stimulation unit 450, a charging/communication unit 470, a coil 480 and/or a battery 490.

Coming back to FIG. 1, the sensor device 100 may further comprise a first fixation mechanism 140 and a second fixation mechanism 150. The first fixation mechanism 140 and the second fixation mechanism 150 may be made of various materials feasible for mechanically fixing the main unit 110 (and thus the sensor device 100) to a narrow organic geometry (e.g., a blood vessel). For example, the material may be durable regarding the organic/aggressive environment, as outlined herein (e.g., the fixation mechanism 140/150 may be made from titanium, a titanium-compound, e.g., nitinol, a titanium coating, etc.). It may also be conceivable that the material may be made from any other suitable metal, plastic, polymer and/or any combination thereof. The fixation mechanisms 140/150 may further comprise various geometries for mechanically fixing the sensor device 100 to a narrow organic geometry (e.g., a blood vessel). For example, the fixation mechanisms 140/150 may comprise a wire geometry, a strip geometry (e.g., a flat shape, a band shape), tines, a screw shape, a hollow tubular shape. In an example, the fixation mechanisms 140/150 may be based on a stent structure.

The sensor device 100 may further comprise a first stimulation portion 145 and a second stimulation portion 155. The stimulation portions 145/155 may comprise any electrically conductive material (e.g., any metal, semiconductor, etc.) to function as an electrode (e.g., for contacting an organic tissue/fluid and coupling electrical energy/power thereto). The surface of the stimulation portions 145/155 may thus function as the interface for coupling the electrical energy/power to the environment. The stimulation portions 145/155 may comprise a porous surface and/or a fractal coating for an improved electrical coupling to an organic tissue/fluid. The fractal coating may be a conductive material in an irregular manner deposited onto the stimulation portions 145/155. This may increase the electrochemically active surface area which may improve the electrical coupling to an organic tissue/fluid (e.g., a blood vessel). This may reduce parasitic electrical effects (e.g., parasitic capacities, parasitic inductances which may impact static, pulsed and/or alternating current based electrical couplings). The first stimulation portion 145 and the second stimulation portion 155 may be electrically/mechanically connected to the main unit 110 and/or elements inside the main unit 110.

The first stimulation portion 145 may be part (e.g., a portion) of the first fixation mechanism 140, and the second stimulation portion 155 may be part (e.g., a portion) of the second fixation mechanism 150. As shown in FIG. 1, the fixation mechanisms 140/150 may be elongated geometries extending from the main unit 110 wherein parts of the fixation mechanisms 140/150 define the stimulation portions 145/155. The stimulation portions 145/155 may be the parts and/or segments of the elongated geometry of the fixation mechanisms 140/150 which may electrically interact with the environment (e.g., parts where electrically conductive material is exposed). Other parts of the fixation mechanisms 140/150 may optionally not interact electrically with the environment. For example, the part extending from the stimulation portion 145 (or 155) to the end of the fixation mechanism 140 (or 150) may not electrically interact with the environment due to its insulating properties regarding the environment. Said insulating part(s) of the elongated geometry of the respective fixation mechanism 140/150 may be electrically insulated due to a coating with an insulating material and/or the part(s) may be made entirely of an insulating material (e.g., a plastic, a polymer, etc.). Hence, only the first stimulation portion 145 and the second stimulation portion 155 may optionally apply an electrical stimulus to the environment. In other examples, the fixations mechanisms are uniformly formed.

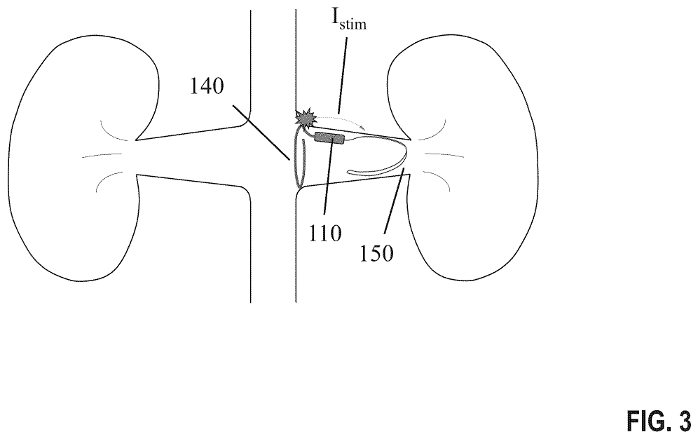

For example, the stimulation portions 145/155 as outlined in FIG. 1 may be configured to function as two electrical terminals which may contact any environment (e.g., a blood vessel, a wall of a blood vessel). Any voltage or current may be applied between the terminals by the sensor device 100 and thus the underlying environment for coupling of an electrical energy/power thereto. The sensor device 100 may thus be configured such that the first stimulation portion 145 and the second stimulation portion may form an electrical circuit through the environment wherein a stimulation current Iₛₜᵢₘ may be conducted through the environment (e.g., due to a voltage difference between the terminals). To control the terminal output, the stimulation portions 145/155 may be electrically connected to one or more elements inside of the main unit 110 (e.g., to a stimulation unit 450) which may control the characteristics of the stimulation current Iₛₜᵢₘ (outlined in detail for FIG. 3/4).

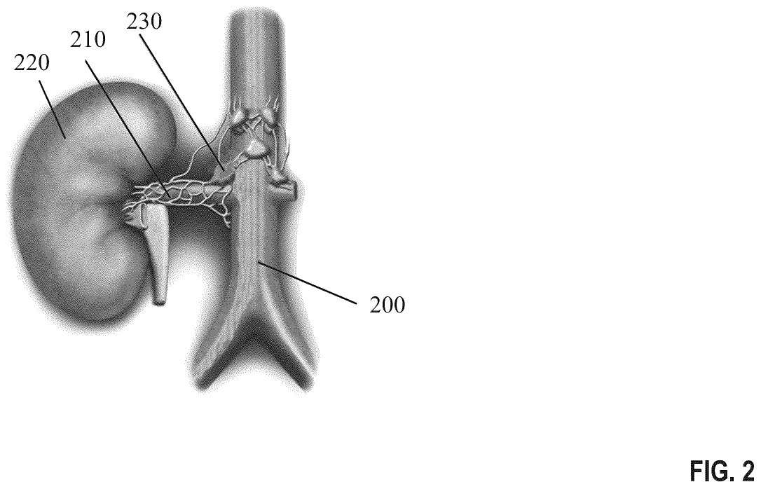

FIG. 2. shows a schematic representation of the anatomy in the kidney area of a human. In particular, a branch of the renal artery 210 of the kidney 220 is shown which is branching off of the aorta 200. The aorta 200 may be the main artery (i.e., a blood vessel) in the human body wherein the renal artery 210 (i.e., a blood vessel) may be an artery that supplies a kidney with blood. A nerve system of the plexus renalis may cover parts of the renal artery 210 and the aorta 200. Inside the nerve system of the plexus renalis may lie an aorticorenal ganglion 230. It is known in the medical field that a stimulation in the area of the aorticorenal ganglion may lead to an increase in blood pressure. The sensor device 100 may be implanted into the renal artery 210 and/or the aorta 200 by an implantation procedure comprising an insertion of a catheter over the inguinal area into the aorta 200. Subsequently, the catheter may be used to place the miniaturized sensor device 100 in the renal artery 210 and/or fixate the sensor device 100 in the renal artery 210, e.g., at a position facing the aorticorenal ganglion and/or close to the aorticorenal ganglion.

FIG. 3 shows a schematic representation of the exemplary embodiment of the sensor device 100 when implanted into a blood vessel, in particular a renal artery 210 in the kidney area of a human (e.g., over an implant procedure as outlined with reference to FIG. 2). The sensor device 100 may be implanted such that the fixation mechanism 140 and the fixation mechanism 150 clamp the sensor device 100 to the renal artery 210 without hindering the flow of blood through the renal artery 210 and/or aorta 200. For example, the first fixation mechanism 140 may comprise a circular or a spiral shape which mechanically attaches to the inner wall of the renal artery 210 such that blood flow may not be blocked by a disturbance in the center of the cross section of the blood vessel. The second fixation mechanism 150 may be configured for the geometry of the renal artery in the vicinity of the kidney wherein the second fixation mechanism 150 may extend over a longer segment in the renal artery than the first fixation mechanism 140. The first stimulation portion 145 and second stimulation portion 155 (not referenced in FIG. 3) may be positioned in the renal artery such that they are in the vicinity of the aorticorenal ganglion 230 (not shown in FIG. 3). For example, the space between the first stimulation portion 145 and second stimulation portion 155 may be matched to the location and/or geometry of the aorticorenal ganglion 230. It may also be conceivable that either the first stimulation portion 145 or the second stimulation portion 155 may be matched to the location and/or geometry of the aorticorenal ganglion 230 (e.g., to its center, edge, etc.). Further, the first stimulation portion 145 and the second stimulation portion 155 may be positioned such that they are close to the wall of the renal artery 210 and/or in direct contact with it. The surface of the stimulation portion 145/155 may thus facilitate an optimal coupling of electrical energy/power (i.e., an electrical excitation) to the renal artery. The coupled electrical energy/power may take on various forms (e.g., implemented by various dynamic and/or static voltage/current inputs to the renal artery) which may be controlled by the sensor device 100. For example, the electrical excitation may be a voltage difference (or a current) applied between the first stimulation portion 145 and second stimulation portion 155 by the sensor device 100 (dynamically and/or statically). This may cause a stimulation current Iₛₜᵢₘ to flow through the renal artery 210 and/or its surrounding tissue/fluid. Hence, depending on the stimulation current Iₛₜᵢₘ, the aorticorenal ganglion (or at least one nerve cell of the aorticorenal ganglion) may be stimulated by the stimulation current Iₛₜᵢₘ such that a blood pressure change is effectuated in the body where the sensor device 100 resides. In another example, the electrical excitation between the first stimulation portion 145 and second stimulation portion 155 may also cause an electrical field through the renal artery 210 and/or its surrounding tissue/fluid. Depending on the electrical field, the aorticorenal ganglion (or at least one nerve cell of the aorticorenal ganglion) may be stimulated by the electrical field such that a blood pressure change is effectuated in the body where the sensor device 100 resides.

FIG. 4. shows a block diagram representing the functional relations of parts of an exemplary embodiment of the sensor device 100. For example, the sensor device 100 may comprise a blood pressure sensor 420, an analog-to-digital converter 430, a microcontroller 440 (alternative also a microprocessor or any other processing unit may be provided), a stimulation unit 450, a charging/communication unit 470, a coil 480 and/or a battery 490. The outlined block diagram shows possible elements of the sensor device 100 when implanted into a blood vessel of a patient (e.g., in a renal artery as outlined in FIG. 3) and a corresponding mode of operation will be outlined below.

For example, the blood pressure sensor 420 of the sensor device 100 may sense the blood pressure 410 of the surrounding blood environment when implanted into a patient. The membrane 130 of the sensor device's main unit 120 may be the sensing element of the blood pressure sensor 420 (as outlined in FIG. 1). The blood pressure sensor 420 may convert the sensed blood pressure to a voltage signal which may correspond to the blood pressure 410 and/or a relative blood pressure change. Subsequently, the voltage signal may be digitized into a digital signal by the analog-to-digital converter 430. The digital signal may be further processed by the microcontroller 440 to determine the blood pressure and/or relative blood pressure change of the environment. In addition, the microcontroller 440 may execute various signal processing steps to the digital signal to further analyze the blood pressure signal (e.g., filtering, evaluating/determining signal characteristics (e.g., a mean value, effective value, creating statistics of the determined blood pressure, comparing with a threshold, etc.)).

Further, the microcontroller 440 may be electrically (e.g., communicatively) coupled to the stimulation unit 450 which may be implemented by or comprise a power-electronic-circuitry (e.g., with a stimulation capacitor, power switches, etc.). The stimulation unit 450 may thus enable shaping a specific electrical excitation 460 wherein the stimulation unit 450 may be electrically coupled to the stimulation portions 145/155 to release the specific electrical excitation 460 to the environment (e.g., as outlined herein). The microcontroller 440 may direct the specific electrical excitation 460 by a certain signaling to the stimulation unit 450, for example.

In an example, the microcontroller 440 may execute various processing steps of a control algorithm to implement a control system for controlling the blood pressure of the patient having the implant. A control input to regulate the blood pressure 410 may be defined by the specific electrical excitation 460 to the blood environment and thus to the at least one nerve cell (e.g., of an aorticorenal ganglion). For example, the control algorithm may control the stimulation unit 450 such that the stimulation unit 450 may release a specific electrical excitation 460 to the blood environment depending on the determined blood pressure 410 to effectuate a change in blood pressure. In addition, the determined blood pressure 410 may be used as feedback for the control system. As an example, the control algorithm may be optimized such that a syncope of the patient having the implant is prevented. For example, the control algorithm may be adapted to hold the blood pressure 410 in a specific range (e.g., between a first predetermined blood pressure of X mmHg and a second predetermined blood pressure of Y mmHg). The specific range may be associated with a medically safe range where the patient is not experiencing a syncope. In another example, the control system (and/or control loop) implemented by the control algorithm may comprise the blood pressure 410 (e.g., the determined blood pressure) as the process variable and/or a single blood pressure value (e.g., of Z mmHg) as a set point of the control system. For example, the control algorithm may implement a P-, I-, PI-, PD- and/or PID-controller.

To illustrate an example of the control input, the specific electrical excitation 460 may be a defined set of one or more current pulses to the blood environment. The defined set may be adapted depending on the currently necessary control input to the system during operation. The defined set of the one more current pulses may be defined by various pulse parameters (e.g., pulse frequency, pulse current amplitude, pulse width, pulse shape, number of applied pulses, duration of the application of the current pulses, pulse duty cycle, etc.). For example, the one or more current pulses may have a frequency of 10 Hz (and/or in the range between 1 Hz and 20 Hz, and/or other values as outlined herein). Further, the one or more current pulses may have a current amplitude of 25 mA (and/or in the range between 10 mA and 40 mA, and/or other values as outlined herein). For example, the control algorithm implemented by the microcontroller 440 may determine at a certain control step that the specific electrical excitation 460 released to the blood environment should be multiple current pulses with a 25 mA current amplitude, a frequency of 10 Hz, a duty cycle of 30%, over a duration of 8 seconds, etc.

FIG. 4 also shows the communication mechanism of the sensor device 100 which may be implemented by a communication unit comprised in the charging/communication unit 470. The communication unit may facilitate the communication to an external device. For example, the communication may be based on wireless communication (e.g., RF, NFC, Bluetooth, etc.). The communication unit may comprise a sender, receiver or transceiver unit for communication with the external device. In another example, the communication unit may be coupled to the microcontroller 440 (e.g., communicatively coupled). This may enable that the external device may communicate with the microcontroller 440 for sending/receiving data. For example, the microcontroller may be programmable over the external device (e.g., by a technician and/or medical personnel). It may also be conceivable that data stored on the microcontroller and/or its peripheral storage medium may be transferred to the external device for external readout of the sensor device's 100 data (e.g., a stored statistical history of blood pressure, system reports, etc.).

FIG. 4 also shows an optional charging mechanism of the sensor device 100. For example, the sensor device 100 may be configured for wireless charging (e.g., inductive charging). The sensor device 100 may thus comprise a rechargeable battery 490 (e.g., a lithium ion battery, a nickel based battery, etc.). As seen in FIG. 4, the sensor device 100 may further comprise a coil 480 which may be excited by an external magnetic field. In an example, the coil 480 may be coupled to a power pick-up unit (not shown). The external magnetic field may be purposefully provided by an external device for charging the sensor device 100. For example, the external device may apply an alternating magnetic field to the coil 480. The magnetic field may thus introduce a current and/or voltage to the coil 480 (i.e., electrical energy) which may be used for electrically charging the rechargeable battery 490 of the sensor device 100. For example, the charging may be supported by a charging unit comprised in the charging/communication unit 470 wherein the charging/communication unit 470 may be coupled to the battery 490 and to the coil 480 (and/or the power pick-up unit). The charging/communication unit 470 may be an electronical unit which may facilitate managing the flow of the electrical energy picked up by the coil 480 to the battery 490 such that the battery 490 is charged in a controlled way (e.g., over a power electronic circuitry, etc.). For, example, the charging unit may be configured to control the charging by activating and/or stopping/halting the charging of the battery 490 when necessary (e.g., when the battery is full the charging may stop and/or the charging may only be activated when the battery is depleted by a certain percentage). In an example, the coil 480 may also be used for communication with the external device to regulate the wireless charging process (e.g., via load shift keying (LSK), etc.) and/or other data communication (e.g., for external readout of the sensor device's 100 data as outlined herein).

In an example, the charging unit and the communication unit of the charging/communication unit 470 may be communicatively coupled or implemented by one element (e.g., by one microcontroller, microprocessor, ASIC). In one respect, this may enable the communication unit to communicate the battery status of the battery 490 to an external device. For example, the communication unit may send a notification and/or alert to the external device if a critical battery status is reached (e.g., low battery power). The notification and/or alert may comprise information that a (e.g., wireless) charging process should be started in a certain time period to prevent an empty battery and/or the depletion of the battery 490. To another respect, the communicative coupling between charging unit and communication unit may be configured for managing the pairing process for the wireless charging process with the external device. For example, the external device may initially communicate to the charging/communication unit 470 that it may provide the electromagnetic field for charging of the sensor device 100. The charging/communication unit 490 may subsequently check if a charging of the battery 490 is possible and/or necessary. Said information may be communicated to the external device which may—depending on the information—start or abort the charging process with the sensor device 100. In another example, the microcontroller 440, the charging/communication unit 470, the charging unit, and/or the communication unit may be implemented by one element (e.g., a microcontroller, aa microprocessor, an ASIC, an embedded system etc.). This may reduce the necessity for multiple hardware or hardware interactions.

In the following, further examples useful for understanding the present invention are listed. The methods may particularly be suitable for treating patients with increased risk for a syncope:
1. Treatment method comprising:
   determining a blood pressure of a patient;
   stimulating at least one nerve cell of the patient, based at least in part on the determined blood pressure.
2. Method of example 1, further comprising:
   implanting a sensor device for determining the blood pressure and/or stimulating the at least one nerve cell.
3. Method of example 1 or 2, wherein the sensor device is configured for implanting into a blood vessel via a catheter.
4. Method of any of examples 1-3, wherein the stimulating comprises stimulating by means for fixing the sensor device to the blood vessel.
5. Method of example 4, wherein the stimulating comprises applying an electrical stimulus to the blood vessel.
6. Method of example 5, wherein applying the electrical stimulus to the blood vessel comprises applying the electrical stimulus in a vicinity of an aorticorenal ganglion.
7. Method of example 5 or 6, wherein the electrical stimulus is adapted to effectuate a change in the blood pressure.
8. Method of any of examples 1-7, further comprising at least one of the following:
   applying one or more stimulations to the at least one nerve cell if the determined blood pressure is below a predetermined threshold;
   applying one or more stimulations to the at least one nerve cell such that the blood pressure is controlled to be between a predetermined first blood pressure and a predetermined second blood pressure;
   stimulating the at least one nerve cell such that a syncope is prevented.

It is noted that the above examples may be combined with further aspects as described herein and details of the examples may also be omitted, as will be understood by the skilled person.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

1. Implantable sensor device comprising:
means for determining a blood pressure;
means for stimulating at least one nerve cell, based at least in part on the determined blood pressure, wherein the sensor device is configured for implanting into a blood vessel, in particular the renal artery,
wherein the sensor device is configured for implantation via a catheter, and wherein the means for stimulating is configured for fixing the sensor device to the blood vessel or the means for stimulating is configured for being electrically coupled to the blood vessel.

2. Implantable sensor device according to any of claim 1,
wherein the means for stimulating is configured to stimulate the at least one nerve cell by applying an electrical stimulus to the blood vessel.

3. Implantable sensor device according to claim 2, wherein the means for stimulating comprises:
a first portion configured for being electrically coupled to a first vessel part of the blood vessel;
a second portion configured for being electrically coupled to a second vessel part of the blood vessel,
wherein the means for stimulating is configured to stimulate the at least one nerve cell by applying an electrical current and/or a voltage between the first portion and the second portion.

4. Implantable sensor device according to claim 1, wherein the at least one nerve cell is a cell of an aorticorenal ganglion.

5. Implantable sensor device according to claim 1, wherein the means for determining comprises a membrane.

6. Implantable sensor device according to claim 1,
wherein the sensor device is configured such that stimulating the at least one nerve cell effectuates a change in the blood pressure.

7. Implantable sensor device according to claim 6, wherein the sensor device is configured to at least one of the following:
apply one or more stimulations to the at least one nerve cell if the determined blood pressure is below a predetermined threshold;
apply one or more stimulations to the at least one nerve cell such that the blood pressure controlled to be between a predetermined first blood pressure and a predetermined second blood pressure;
stimulate the at least one nerve cell such that a syncope is prevented.

8. Implantable sensor device according claim 7, wherein the sensor device is configured for wireless charging.

9. Implantable sensor device according to claim 8, wherein the sensor device is configured for communication with an external device.

10. Method carried out by an implantable sensor device comprising the following steps:
determining a blood pressure;
stimulating at least one nerve cell, based at least in part on the determined blood pressure, wherein the sensor device is configured for implanting into a blood vessel.

11. Computer program comprising instructions to perform a method of claim 10, when the instructions are executed by an implantable sensor device.
